(19) United States
(12) Patent Application Publication
(10) Pub. No.: US 2026/0259186 A1
(43) Pub. Date: Sep. 3, 2026

(54) AIR QUALITY MONITORING SYSTEM AND METHOD

(71) Applicant: Antrum, Inc., Grand Rapids, MI (US)

(72) Inventors: Ryan Mulder, Jenison, MI (US); Taylor Groll, Byron Center, MI (US); Randall Vandermate, JR., Zeeland, MI (US); Micah TerHaar, Byron Center, MI (US); Adam Donovan, Ada, MI (US)

(73) Assignee: Antrum, Inc., Grand Rapids, MI (US)

(21) Appl. No.: 19/554,389

(22) Filed: Mar. 2, 2026

Related U.S. Application Data
(60) Provisional application No. 63/764,836, filed on Feb. 28, 2025.

(51) Int. Cl. G01N 33/00; F24F 11/00; F24F 110/10; F24F 110/20; F24F 110/30; F24F 110/40; F24F 110/64; F24F 110/66; F24F 110/70; F24F 110/72; F24F 110/74; G01B 7/30

(52) U.S. Cl. CPC G01N 33/0031; F24F 11/0001; G01B 7/30; F24F 2110/10; F24F 2110/20; F24F 2110/30; F24F 2110/40; F24F 2110/64; F24F 2110/66; F24F 2110/70; F24F 2110/72; F24F 2110/74

(57) ABSTRACT

An air quality monitoring system is provided. The system generally comprises a controller, a multiplexor, and a sensor pack having at least one sensor configured to measure an air quality metric of the sample. The multiplexor is configured to receive samples from the plurality of different rooms at a plurality of multiplexor inlets. The multiplexor may further include a rotatable core that defines a first core inlet. The multiplexor and the sensor pack collectively define a sensor airflow line that fluidly couples the multiplexor inlet associated with the selected room, the first core inlet, and a remote test environment within the sensor pack. The multiplexor further comprises a power unit in electrical communication with the rotatable core and that, when instructed by the controller, rotates the rotatable core between positions thereby selecting the alignment of the sensor airflow line with the multiplexor inlet of the selected room.

### CROSS REFERENCE TO RELATED APPLICATIONS

This application claims the benefit of U.S. Provisional Application No. 63/764,836, filed on Feb. 28, 2025, which is hereby incorporated by reference in its entirety.

### TECHNICAL FIELD

The present disclosure generally relates to air monitoring systems and, more particularly, to air monitoring systems utilizing multipoint air sampling.

### BACKGROUND

Building air supply and control systems are becoming increasingly important due to increases in energy costs. In various building ventilation systems (e.g., HVAC systems), it may be beneficial to improve data acquisition for each room or regions within rooms or spaces of the building. Such systems, while beneficial in a variety of environments, are particularly important in the laboratory context and health care (hospital) context. The disclosure provides for a variety of improvements in air sampling and air quality control for building ventilation and control systems.

### SUMMARY

An air quality monitoring system configured to detect an air quality metric of a plurality of air samples delivered from a plurality of different regions and an associated method for control thereof is disclosed and described. The system generally comprises a multiplexor, a sensor pack, and a controller in electrical communication with each of the multiplexor and the sensor pack.

The sensor pack comprises a sensor pack housing defining a sensor pack inlet and a sensor pack outlet, and at least one sensor disposed within the housing, wherein at least one sensor configured to measure an air quality metric of the selected air sample.

The multiplexor is configured to receive a plurality of air samples from each of the plurality of different regions or rooms. The multiplexor may include a housing having a housing base that defines a multiplexor sensor outlet and a purge outlet. The multiplexor housing may further include a housing cover that defines a plurality of multiplexor inlets in fluid communication with the plurality of volumetric regions or rooms, wherein the multiplexor defines at least a first multiplexor inlet that corresponds to a measured volumetric region and a second multiplexor inlet that corresponds to a primed volumetric region. The multiplexor may further include a rotatable core and a bypass structure, wherein the rotatable core defines a first core outlet and a second core outlet and the bypass structure defines a bypass inlet and a bypass outlet.

Accordingly, the multiplexor defines a purge airflow line that fluidly couples the second multiplexor inlet, the second core inlet, the bypass inlet, the bypass outlet, and the purge outlet, such that the purge airflow line conveys the air sample from the primed volumetric region to a building ventilation or HVAC system.

The multiplexor and the sensor pack collectively define a sensor airflow line that fluidly couples the first multiplexor inlet, the first core inlet, the multiplexor sensor outlet, the sensor pack inlet that conveys the air sample from the measured volumetric region to a remote test environment within the sensor pack that simulates the environment with representative air quality conditions to that of the measured volumetric region.

The multiplexor further comprises a power unit in electrical communication with the rotatable core and that is configured, when instructed by the controller, to rotate the rotatable core between a plurality of positions thereby selecting the alignment of the sensor airflow line with the multiplexor inlet of a selected room or region for evaluation or measurement.

### BRIEF DESCRIPTION OF THE DRAWINGS

The embodiments set forth in the drawings are illustrative and exemplary in nature and not intended to limit the subject matter. The following detailed description of the illustrative embodiments can be understood when read in conjunction with the following drawings, where like structure is indicated with like reference numerals and in which:

FIG. 1 is a block diagram of a building demonstrating a plurality of rooms or test regions incorporating the air quality monitoring system of the present disclosure.

FIG. 2 is a block diagram of a building demonstrating a plurality of rooms or test regions incorporating the air quality monitoring system of the present disclosure.

FIG. 3 is a schematic diagram of the control air quality monitoring system of the present disclosure.

FIG. 4 is a detailed schematic diagram of the sample return unit of the air quality monitoring system of the present disclosure.

FIG. 5A is a schematic, top, perspective view of an example improved multiplexor used within the air quality monitoring system of the present disclosure.

FIG. 5B is a schematic, top view of an example improved multiplexor used within the air quality monitoring system of the present disclosure.

FIG. 5C is a schematic, exploded, top, perspective view of an example improved multiplexor used within the air quality monitoring system of the present disclosure.

FIG. 5D is a cut-away view of an example improved multiplexor used within the air quality monitoring system of the present disclosure, showing the internal structure and air paths of the same.

FIG. 5E is a cut-away view an example improved multiplexor used within the air quality monitoring system of the present disclosure, showing the internal structure and air paths of the same as well as the cumulative airflow diagram for air passing therethrough.

FIG. 6A is a schematic, top perspective view of an example, fully assembled sensor pack of the air quality monitoring system of the present disclosure.

FIG. 6B is a schematic, side view of an example, fully assembled sensor pack of the air quality monitoring system of the present disclosure, having the external housing thereof removed.

FIG. 6C is a schematic top, cross-sectional view of the fully assembled sensor pack of the air quality monitoring system of the present disclosure as shown in FIGS. 6A and 6B taken along line 6C-6C.

FIG. 6D is a schematic, top, cross-sectional view of the fully assembled sensor pack of the air quality monitoring system of the present disclosure as shown in FIGS. 6A and 6B taken along line 6D-6D.

FIG. 6E is a schematic, bottom, cross-sectional view of the fully assembled sensor pack of the air quality monitoring system of the present disclosure as shown in FIGS. 6A and 6B taken along line 6E-6E.

FIG. 6F is a schematic, bottom, cross-sectional view of the fully assembled sensor pack of the air quality monitoring system of the present disclosure as shown in FIGS. 6A and 6B taken along line 6F-6F.

### DETAILED DESCRIPTION OF EMBODIMENTS

While the present disclosure may be described with respect to specific applications or industries, those skilled in the art will recognize the broader applicability of the disclosure. Those having ordinary skill in the art will recognize that terms such as “a”, “an”, “the”, “at least one”, and “one or more” are used interchangeably to indicate that at least one of the items is present. A plurality of such items may be present unless the context clearly indicates otherwise. All numerical values of parameters (e.g., of quantities or conditions) in this specification, unless otherwise indicated expressly or clearly in view of the context, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. In addition, a disclosure of a range is to be understood as specifically disclosing all values and further divided ranges within the range.

The terms “comprising”, “including”, and “having” are inclusive and therefore specify the presence of stated features, steps, operations, elements, or components, but do not preclude the presence or addition of one or more other features, steps, operations, elements, or components. Orders of steps, processes, and operations may be altered when possible, and additional or alternative steps may be employed. As used in this specification, the term “or” includes any one and all combinations of the associated listed items. The term “any of” is understood to include any possible combination of referenced items, including “any one of” the referenced items. The term “any of” is understood to include any possible combination of referenced claims of the appended claims, including “any one of” the referenced claims.

Features shown in one figure may be combined with, substituted for, or modified by, features shown in any of the figures. Unless stated otherwise, no features, elements, or limitations are mutually exclusive of any other features, elements, or limitations. Furthermore, no features, elements, or limitations are absolutely required for operation. Any specific configurations shown in the figures are illustrative only and the specific configurations shown are not limiting the claims or the description.

For consistency and convenience, directional adjectives are employed throughout this detailed description corresponding to the illustrated embodiments. Those having ordinary skill in the art will recognize that terms such as “above”, “below”, “upward”, “downward”, “top”, “bottom”, etc., may be used descriptively relative to the figures, without representing limitations on the scope of the invention, as defined by the claims. Any numerical designations, such as “first” or “second” are illustrative only and are not intended to limit the scope of the disclosure in any way.

The term “longitudinal”, as used throughout this detailed description and in the claims, refers to a direction extending a length of a component. In some cases, a component may be identified with a longitudinal axis as well as a forward and rearward longitudinal direction along that axis. The longitudinal direction or axis may also be referred to as an anterior-posterior direction or axis.

The term “transverse”, as used throughout this detailed description and in the claims, refers to a direction extending a width of a component. The transverse direction or axis may also be referred to as a lateral direction or axis or a mediolateral direction or axis.

The term “vertical”, as used throughout this detailed description and in the claims, refers to a direction generally perpendicular to both the lateral and longitudinal directions.

In addition, the term “proximal” refers to a direction that is nearer a center of a component. Likewise, the term “distal” refers to a relative position that is further away from a center of the component. Thus, the terms proximal and distal may be understood to provide generally opposing terms to describe relative spatial positions.

Modern heating, ventilation, and air conditioning (HVAC) systems may provide for independent control of various regions or rooms within a building. In this way, climate control and air quality may be monitored and controlled for each region or room. In addition to the benefits of improved comfort and air quality, systems may also be configured to reduce energy consumption. However, there are a variety of challenges that may be associated with implementing such systems on a large scale while maintaining economical HVAC control solutions. The following detailed description provides for an air quality monitoring system and associated methods that are effective, economical, and provide ease of routine maintenance.

Accordingly, the disclosure may provide for the improved operation of an air quality monitoring system as illustrated by the following illustrative examples.

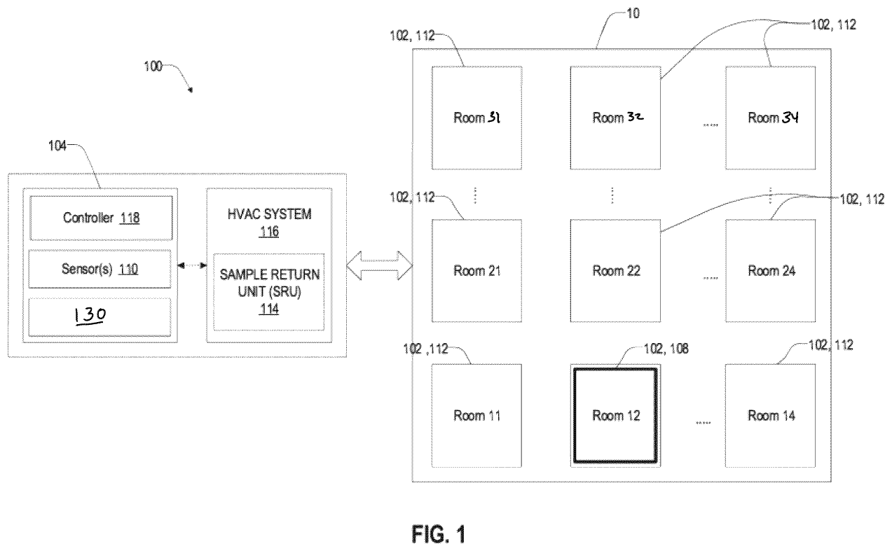

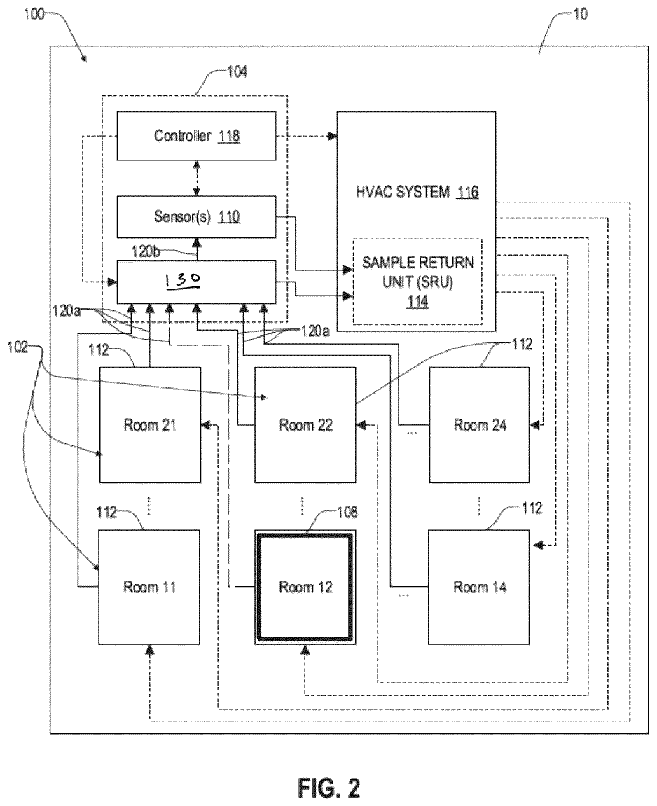

Referring to the drawings, wherein like reference numerals refer to like components throughout the several views, an air quality monitoring system 100 and the component parts thereof are shown and described. Referring more particularly to FIGS. 1 and 2, block diagrams of a building 10 having an air quality monitoring system 100 are shown. The air quality monitoring system 100 may be configured to selectively measure at least one air quality metric or characteristic from a plurality of areas or rooms of the building 10. For example, the system 100 may be configured to monitor various volumetric regions of a single room and/or monitor the conditions of each of a plurality of rooms 102 or regions (e.g., rooms 11, 12, 14, 21, 22, 24, 31, 32, 34 shown in FIG. 1). The plurality of rooms 102, regions, or areas of the building 10 may correspond to regions or volumetric spaces within a commercial building, residential building, multi-purpose building, or another defined enclosure or volumetric enclosure that is envisioned for human occupancy, such as an automobile, airplane, etc. The disclosure may provide for various novel aspects configured to limit the installation, operating, and maintenance cost of the system 100, and may also provide for improved operational performance. Though the rooms 102 may correspond to various regions, areas, sections, portions, etc. of a building or volumetric space, the term “room” or “rooms” are used for clarity herein.

In one example embodiment, the system 100 may comprise an air sampling device 104, a multiplexor 130, a sensor pack 110, and a controller 118. The air sampling device 104 may correspond to a scalable monitoring device configured to sample the air from each of the plurality of rooms 102 via direction or signal from the controller 118. For example, the system 100 may comprise a faceplate or duct probe disposed in each respective room and operatively coupled to the multiplexor 130 at a respective multiplexor inlet 106 by a room-specific tube or supply line 120. The tubes or supply lines 120 extend from the respective air sampling device 104 in each room to the multiplexor 130. The tubes or supply lines 120 may comprise 0.25-inch diameter polymer tubing, which in one example may be comprised of a fluorinated ethylene propylene material.

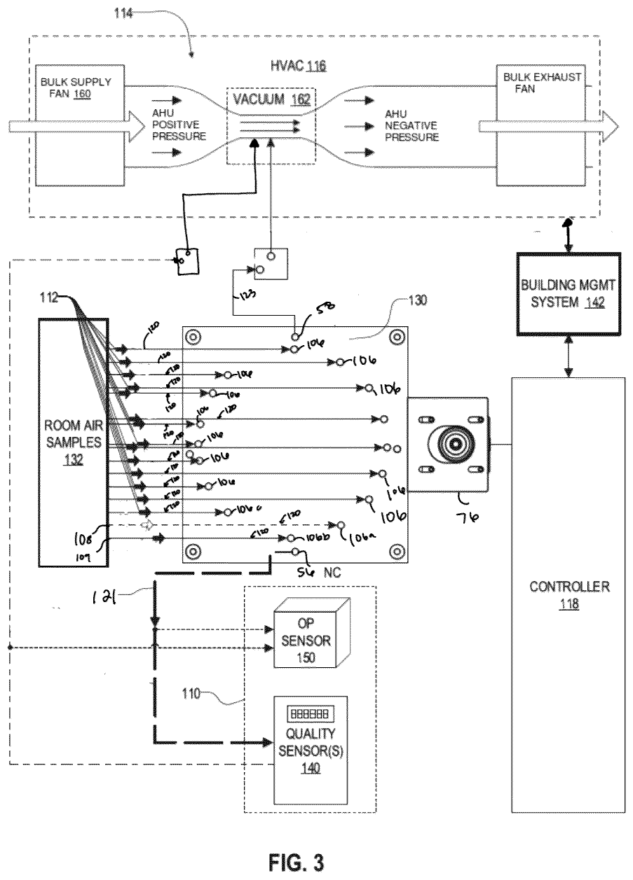

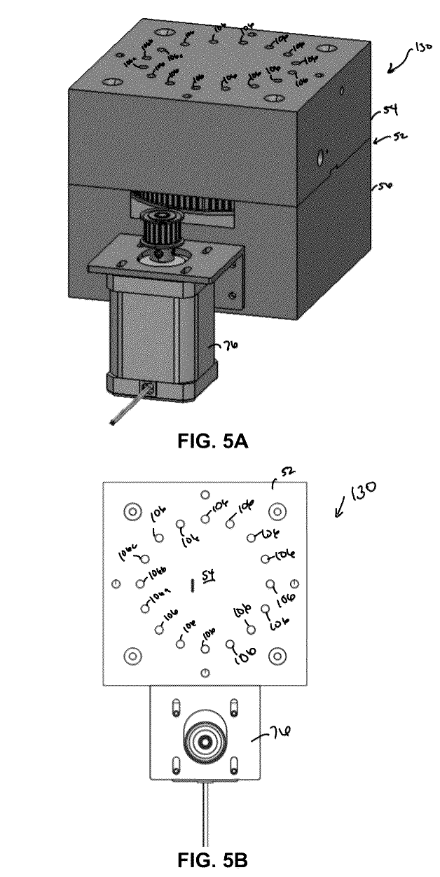

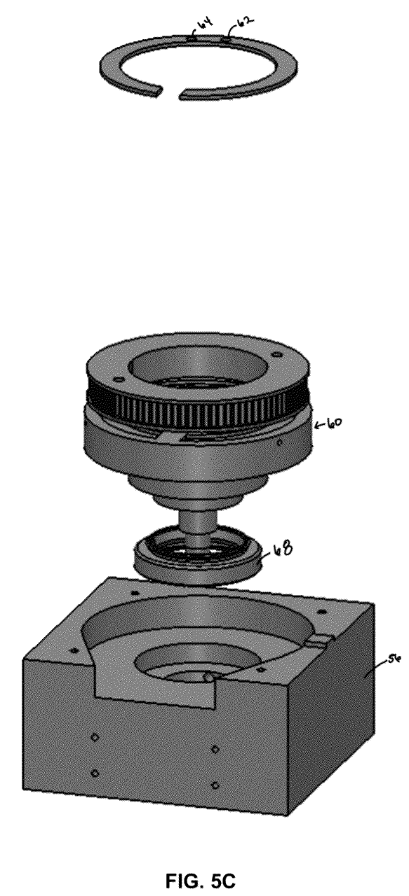

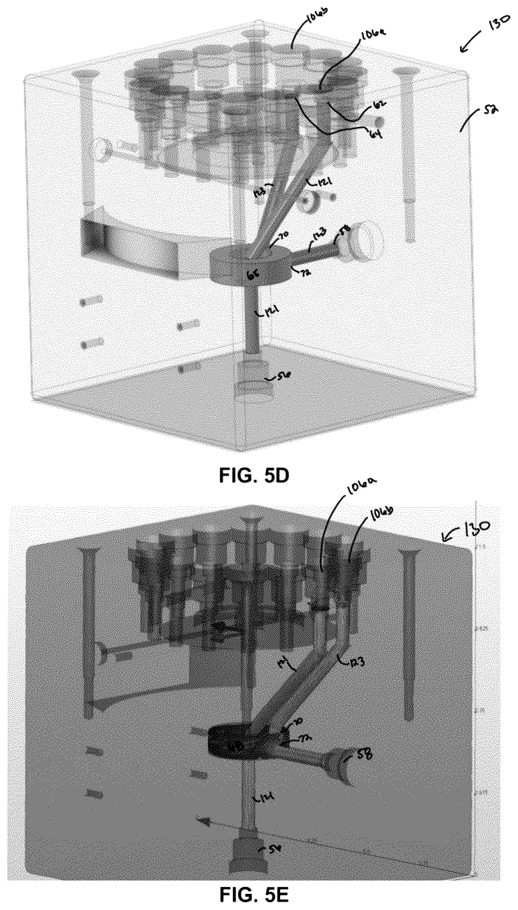

The system 100 may further comprise a multiplexor 130 (FIGS. 3 and 5A-5E). Referring to FIG. 3, the multiplexor 130 is configured to receive multiple air quality samples or inputs corresponding to each of the plurality of rooms or volumetric regions 102 at a plurality of multiplexor inlets 106. Said another way, the supply line to tube 120 corresponding to each respective room 102 (e.g. rooms 11, 12, 14, 21, 22, 24, 31, 32, 34) terminates at one of the plurality of multiplexor inlets 106, such that each room is monitored independently, but the multiplexor 130 via the controller 118 selects the room for sensing or measurement or a measured volumetric region 108 (e.g., room 12) wherein the air sample is conveyed to the one or more sensors housed in the sensor pack 110 via a sensor airflow line 121, a next in line room for evaluation or a primed channel volumetric region 109 (e.g. room 14) wherein the air sample is purged via a purge airflow line 123. In one example, the multiplexor 130 is a 16×2 multiplexor in that it has sixteen (16) total multiplexor inlets 106 (corresponding to sixteen (16) rooms 102) and two outlets, discussed in more detail herein below.

Referring to the FIGS. 5A-5E, the multiplexor 130 is shown in greater detail. The multiplexor 130 in its entirety may be formed of a chemically inert material. The multiplexor 130 comprises a housing 50 having a base 52 and a cover 54. The cover 54 defines the plurality of multiplexor inlets 106. The number of multiplexor inlets 106 corresponds to the number of rooms 102 connected thereto, as shown by example in FIGS. 3 and 5A-5E, sixteen (16) inlets are shown. In any such embodiment, the plurality of multiplexor inlets 106 may include at least a first multiplexor inlet 106a corresponding to a first room or a measured volumetric region 108 and a second multiplexor inlet 106b corresponding to a second room or next in line room for evaluation or a primed channel volumetric region 109. In another example, the plurality of multiplexor inlets 106 may also include a third multiplexor inlet 106c. In one example, the cover 54 may be comprised of a Polytetrafluoroethylene (PTFE) material. The base defines a multiplexor sensor outlet 56 and a purge outlet 58. In one example, the base 58 may be comprised of an acrylonitrile butadiene styrene (ABS) material.

The multiplexor 130 may further comprise a rotatable core 60 defining a first core inlet 62 and a second core inlet 64. The rotatable core 60 may be comprised of a Polytetrafluoroethylene (PTFE) material. The multiplexor 130 may further comprise a bypass structure 68 which defines a bypass inlet 70 and a bypass outlet 72. A sensor airflow line 121 is defined as a tube that fluidly couples the first multiplexor inlet 106a, first core inlet 62, and the sensor outlet 56 which transports an air sample from the measured volumetric region to the sensor pack 110 and eventually, as detailed herein below, through the sensor pack inlet 78, the air channel 126, and the sensor pack outlet 80. A purge airflow line 123 is defined as a tube that fluidly couples the second multiplexor inlet 106b, second core inlet 64, the bypass inlet 70, the bypass outlet 72, and the purge outlet 58, such that the purge airflow line 123 transports an air sample from the next in line room for evaluation or a primed channel volumetric region 109 to the building ventilation or HVAC system 116.

A power unit 76, such as an electric motor, may be in electrical communication with the rotatable core 60 and the controller 118. As such, the controller 118 is configured to output a control signal to the power unit 76 to rotate the rotatable core 60 to a first position, such that the first core inlet 62 is aligned with the first multiplexor inlet 106a, that is associated with the room for sensing or measurement or the measured volumetric region 108 (e.g., room 12), such that the air sample corresponding to the measured volumetric region 108 (e.g., room 12), is conveyed to the sensor pack 110 via the sensor airflow line 121. In this way, in the first position, the second multiplexor inlet 106b is aligned with the second core inlet 64, such that the air sample corresponding to the primed volumetric region 109 (e.g. room 14) is discarded via the purge airflow line 123. In such an example, the third multiplexor inlet 106c corresponds to an idle room 112 (e.g., room 21) is stopped or blocked at the respective multiplexor inlet 106.

Said another way, while drawing the room air sample from the first room or the measured volumetric region 108 (e.g. room 12) for direction to the sensor pack 110 via the sensor airflow line 121 and drawing the room air sample from the second or primed channel room 109 (e.g. room 14) for direction to a purge airflow line 123, the air sampling device 104 may be configured to draw air or communicate air from the idle rooms 112 of the plurality of rooms 102 but such air samples are stopped or blocked at the respective multiplexor inlet 106.

After the controller 118 receives readings or data from the respective sensors 140, 150 in the sensor pack 110 related to the measured volumetric region 108 (e.g., room 12), the controller 118 is configured to output a control signal to the power unit 76 to rotate the rotatable core 60 to a second position, such that the first core inlet 62 is aligned with the second multiplexor inlet 106b, that is associated with the new room selected for sensing or measurement or the measured volumetric region 108 (e.g., room 14), such that the air sample corresponding to the measured volumetric region 108 (e.g., room 14), is conveyed to the sensor pack 110 via the sensor airflow line 121. In this way, in the second position, the third multiplexor inlet 106c is aligned with the second core inlet 64, such that the air sample corresponding to the new room to be primed volumetric region 109 (e.g. room 21) is discarded via the purge airflow line 123. The controller 118 is configured to continue to instruct the power unit 76 to control the rotation of the rotatable core 60 until all rooms 102 have been primed and then evaluated.

The multiplexor 130 may further comprise a magnetic encoder configured to evaluate the relative position of the rotatable core 60 and transmit a core position signal to the controller 118. The multiplexor 130 may further comprise an absolute position sensor. The absolute position sensor comprises an alignment bore that passes through the cover 54 and the rotatable core 60 and an infrared beak beam sensor. In operation, the infrared beam of the break beam sensor shines through the bore in the cover 52 and the bore in the core 60 when the same are aligned. When the bore in the cover 52 and the bore in the core 60 are misaligned, the infrared beam is broken indicating misalignment.

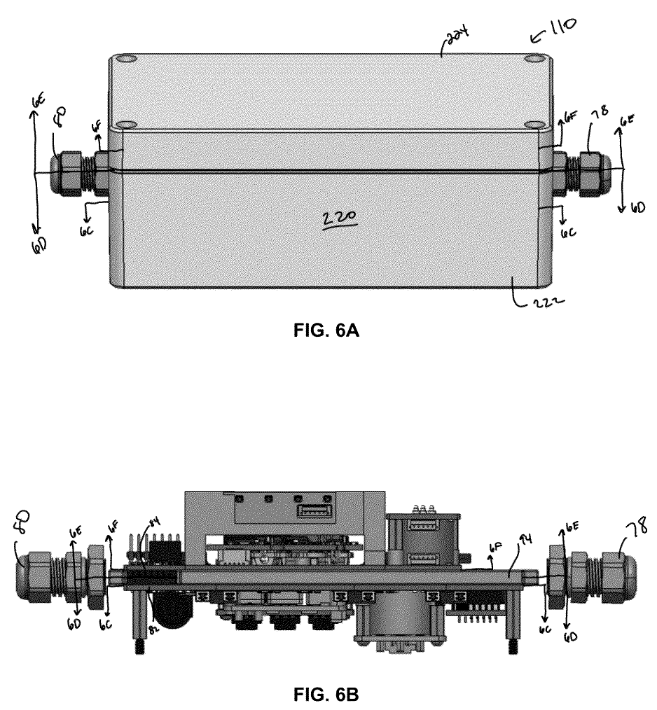

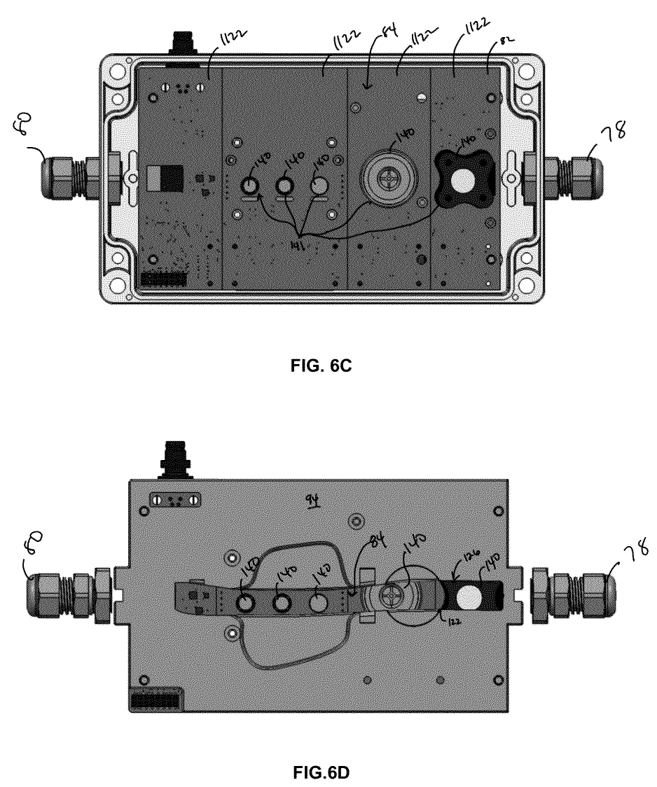

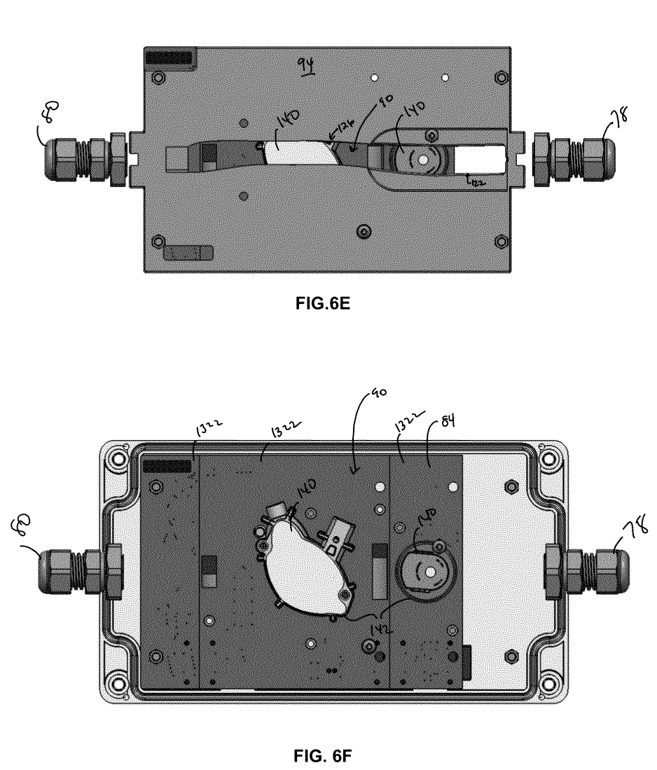

The system 100 may further comprise at least one sensor pack 110 as shown in FIGS. 6A-6F. The sensor pack 110 may comprise a housing 220 that defines a sensor pack inlet 78 in fluid communication with the multiplexor sensor outlet 56 and a sensor pack outlet 80. The sensor pack housing 220 may further comprise a base 222 and a cover 224. The sensor pack housing 220 in its entirety may be formed of a chemically inert material. In one example, the sensor pack housing 220 may be comprised of an acrylonitrile butadiene styrene (ABS) material. Each of the sensor pack inlet 78 and the sensor pack outlet 80, and the air channel 126 (detailed herein below) disposed therebetween are positions on the sensor airflow line 121.

The sensor pack 110 may comprise one or more operation sensors 150. The operation sensors 150 may correspond to one or more forms of air flow rate sensors or flow meters, differential pressure transmitters, and pressure sensors, etc. The sensor pack 110 may comprise at least one air quality sensor 140 within the housing 220. The at least one air quality sensor 140 may correspond to a variety of types of sensors configured to measure one or more properties or air quality parameters of the room air samples 132. In some embodiments, the at least one air quality sensor 140 may be configured to detect a condition or change in condition in each of the room air samples 132. For example, the one or more air quality sensors 140 may correspond to one or more forms of temperature sensors, volatile organic compound (VOC) sensors, particulate matter sensors, relative humidity sensors, carbon dioxide sensors, carbon monoxide sensors, ozone sensors, ammonia sensors, formaldehyde sensors, and the like. Additionally, the one or more air quality sensors 140 may correspond to electro-chemical sensors that may be configured to detect one or more allergens or particulate materials suspended in the air within the measured volumetric region 108 or room.

In some examples, the operation sensor 150 and the at least one air quality sensor 140 are organized as a first plurality of sensors 141 and a second plurality of sensors 142. The first plurality of sensors 141 may be disposed on a first sensor board 82 having a first board sensing surface 84 and a first board auxiliary surface 86. The first sensor board 82 may comprise a plurality of board segments or modules 1122, wherein each board segment or module 1122 comprises one of the sensors of the first plurality of sensors 141. Each of the board segments or modules 1122 are selectively couplable and removable from the other board segments or modules 1122 and may be coupled to each other via male/female connectors known in the art. Modularity of the first sensor board 82 allows for each of removal, replacement, upgrade, or calibration of individual sensors, without the need to replace the entire sensor board 82 or sensor pack 110.

The second plurality of sensors 142 may be disposed on a second sensor board 88 having a second board sensing surface 90 and a second board auxiliary surface 92. The second sensor board 88 may comprise a plurality of board segments or modules 1322, wherein each board segment or module 1322 comprises one of the sensors of the second plurality of sensors 142. Each of the board segments or modules 1322 are selectively couplable and removable from the other board segments or modules 1322 and may be coupled to each other via male/female connectors known in the art. Modularity of the second sensor board 88 allows for each of removal, replacement, upgrade, or calibration of individual sensors, without the need to replace the entire sensor board or sensor pack 110.

It is contemplated that blank board segments or modules 1122, 1322, which do not have a coupled sensor 140, may be used as placeholders to fill space within the sensor pack housing 220, allowing for future sensor expansion or ensuring the structural integrity of the respective sensor board 82, 88.

The sensor pack 110 further includes a substrate layer 94 which defines an air channel 126 therein that extends from the sensor pack inlet 78 to the sensor pack outlet 80. The air channel 126 is designed as a substantially linear path for air to flow from the sensor pack inlet 78 to the sensor pack outlet 80, such that no turbulence of airflow is created wherein particles or sensed items may be dropped from the respective air sample 132, nor velocity slowed within the air channel 126.

The substrate layer 94 is disposed between the first sensor board 82 and the second sensor board 88 to create space therebetween to allow the respective air sample to move through the sensor pack 110 from the sensor pack inlet 78 to the sensor pack outlet 80. The substrate layer 94 may be a unitary piece or may be comprised of a plurality of stacked spacer plates.

In one example embodiment, as shown in FIGS. 6B-6E, the sensor pack 110 may be assembled from the bottom up. Said another way, the first auxiliary surface 86 is disposed in contact with the housing base 222. The substrate layer 94 is disposed in contact with the first sensing surface 84 and the second sensing surface 90, such that each of the first plurality of sensors and the second plurality of sensors is aligned with the air channel 126. The second board auxiliary surface 92 is disposed in contact with the housing cover 224.

Accordingly, a sealed enclosure may be formed within the sensor pack 110 via the air channel 126, such that a remote test environment 122 is created. In operation, the multiplexor 130 may continuously cycle sampled air from each of the rooms 102 and selectively deliver the air from the selected room 108 into the remote test environment 122 formed within the air channel 126. In this way, the system 100 may provide for a simulated environment with representative air quality conditions to that of the selected room 108 and each of the rooms 102 in a remote location.

The sealed enclosure of the remote test environment 122 may prevent contamination or seepage from a local environment from entering the sealed enclosure and interrupting or otherwise limiting accuracy of the representation of the air conditions within the remote test environment 122 relative to those in the selected room 108.

Preventing seepage from the local environment may be of particular importance for the system 100 in implementations that utilize the sample return unit 114 in the form of a Venturi vacuum device (detailed hereinbelow) for similarly low energy systems, which may derive differential pressure from air flow controllers for the HVAC system 116 of the building. For example, in systems that utilize sample return units 114 that are only operable to generate limited differential pressure (e.g., less than two (2) inches of water or five (5) mbar), seepage from the local environment may form a significant portion of the air and related contaminates entering the remote test environment 122. Accordingly, in order to direct sufficient air from the selected room 108 into the remote test environment 122 via the sensor airflow line 121, the sealed enclosure comprising the remote test environment 122 should be effectively sealed, particularly in systems that are optimized for efficient operation with limited flow rates passing through the air channel 126.

In some implementations, in order to ensure the integrity of the remote test environment 122, the system 100 may further comprise an operation sensor 150 that is defined as a differential pressure sensor. The differential pressure sensor may be configured to detect the pressure between the sensor pack inlet 78 and sensor pack outlet 80 in order to ensure that the sealed enclosure has not been breached or otherwise compromised. Accordingly, the differential pressure sensor may be in communication with the controller 118, such that the controller 118 may monitor pressure across the remote test environment 122 and thereby test the integrity of the sealed enclosure. As demonstrated, the pressure sensor is configured to detect an external pressure differential in order to ensure that the integrity of the sealed enclosure and the connections (e.g., the sensor pack inlet 78 and sensor pack outlet 80), and any external fittings are maintained such that the environment within the sealed enclosure is isolated from the environment outside the sensor pack 110. In this way, the system may diagnose potential leaks and various system operating defects in response to changes detected in the differential pressure.

More generally, an operation sensor 150 that is defined as an airflow sensor or velocity sensor may allow the system 100 to determine an airflow profile of a plurality of air samples 132 delivered from a plurality of different regions or rooms 102, such that the system may detect compromised tubing 120 (e.g., punctured, cut, kinked, clogged, etc.) based on variations in airflow rate measurements. More particularly, the test apparatus and associated method of the present disclosure may aid in the detection of tubing that has become compromised by monitoring variations in the flow rate of air samples from the respective rooms or regions by comparing real-time flow measurements against stored baseline data, thereby maintaining the integrity of air quality assessments, and ensuring reliable operation of the monitoring system.

In an exemplary operation of the system 100, the system 100 may detect compromised tubing 120. The method may begin by initiating operation for rooms 102 or regions of the building 10. The operation may be initiated by the controller 118, which is configured to output a control signal to the power unit 76 to rotate the rotatable core 60 to a first position, such that the first core inlet 62 is aligned with the first multiplexor inlet 106a, that is associated with the room for sensing or measurement or the measured volumetric region 108 (e.g., room 12), such that the air sample corresponding to the measured volumetric region 108 (e.g., room 12), is conveyed to the sensor pack 110 via the sensor airflow line 121.

Once initiated, the rotatable core 60 is disposed in the first position, the air supplied to the sensor airflow line 121 and the remote test environment 122 within the sensor pack 110 from the selected room 108 may be purged from air channel 126 out through the sensor pack outlet 80 for a predetermined time period or sensor airflow line purge time Tₚ. During the sensor airflow line purge time Tₚ, the air flow rate sensor may measure a first air flow rate reading of the selected room 108. The first air flow rate reading may be stored in a memory or database. The air flow rate sensor may measure a second air flow rate of the selected room 108 at a later point in time. If the second air flow rate differs from the first air flow rate, the controller 118 may take another reading to verify the measurements. If all subsequent airflow rate measurements show a consistent difference from the initial measurement, the controller 118 may determine that the suction has changed and update the stored measurements accordingly. However, if the measurements are not similarly different, the controller 118 may identify that the tubing is compromised, indicating a potential issue such as a puncture, cut, kink, clog, or other mechanical failure. The controller 118 may sequentially monitor the air flow rate of each of the regions or rooms 102 in fluid communication with sealed enclosure 122.

Referring back to FIGS. 6C-6F, to avoid interference among the sensors 140, 150 in the air channel 126, the arrangement of the sensors 140, 150 may be provided in a sequential order or along unique or otherwise non-overlapping flow paths within the air channel 126. More generally, in cases where a temperature sensor is included in the sensor pack 110, the temperature sensor may be arranged on the respective board segment 1122, 1322 that is closest to the sensor pack inlet 78, upstream of any sensors that may disrupt the temperature reading, particularly those that generate heat or otherwise could adjust a temperature of air supplied to the temperature sensor along the flow path from sensor pack inlet 78 to sensor pack outlet 80.

Similarly, the operation sensor 150 may be arranged on the respective board segment 1122, 1322 that is closest to the sensor pack inlet 78. In this configuration, the operation sensor 150 may be configured to identify if the air flow, in terms of velocity and differential pressure, provided to the sensor pack 110 is sufficient to measure the air quality parameter of the selected room with the air quality sensors 140. Said another way, the operation sensor 150 may be configured to identify a change in pressure over the operating life or maintenance cycle of the air sampling system 100, such as a puncture, cut, clog, bend, or kink in the tubing of the air sample supply lines or tubes 120 for one or more of the plurality of rooms 102 between the air sampling device 104 in each room 102 and the respective multiplexor inlet 106 to the multiplexor 130.

In one example embodiment, the system 100 may function such that the one or more air quality sensors 140 may be configured to communicate one or more measurements or other data to the controller 118. The controller 118 may process the one or more measurements or other data to identify air quality conditions or metrics for each of the plurality of rooms 102. The measurements communicated to the controller 118 may be utilized to determine an appropriate action to improve the air quality for a specific room of the plurality of rooms 102. In this way, the controller 118 may identify a condition or change in condition in one or more air quality parameters. Based on the identified condition or change, the controller 118 may communicate a ventilation control signal configured to induce a building management system (BMS) 142 to take corrective action. In response to the ventilation control signal, the BMS 142 may control the HVAC system 116 to provide ventilation, heat, and/or cooled air to a specific room or group of rooms of the plurality of rooms 102.

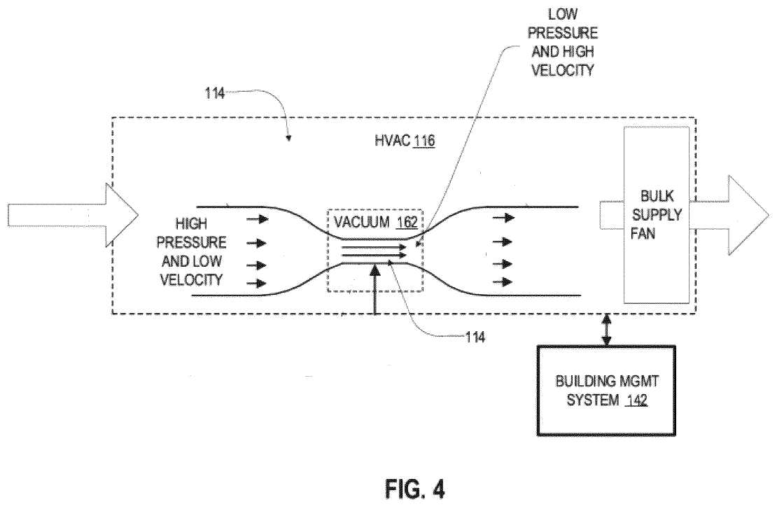

Referring back to FIGS. 1-4, the system 100 may further comprise an air transport motivator or sample return unit 114. The sample return unit 114 may correspond to an energy efficient air transport unit configured to function without a need for direct electrical power. In such embodiments, the sample return unit 114 may be configured to generate suction to draw the room air samples from each of the plurality of rooms 102 via a bulk supply fan of an existing heating, ventilation, and air conditioning (HVAC) system 116 of the building 10. For example, the sample return unit 114 may correspond to a Venturi vacuum device configured to utilize differential pressure derived from the bulk supply fan of the HVAC system 116. The HVAC system 116 may be configured to provide heating, ventilation, and air conditioning to each of the rooms 102.

In one exemplary embodiment utilizing the Venturi vacuum device, the only energy usage of the sample return unit 114 may be a small parasitic energy drawn from the bulk supply fan of the HVAC system 116. For example, the bulk supply fan may consume a small and potentially negligible increased power due to the energy usage of the sample return unit 114. The change in power usage may be insignificant, particularly when the air sampling system 100 is implemented in large buildings having multiple rooms and high-volume HVAC systems 116. In this way, the air sampling system 100 may improve efficiency while limiting components and related maintenance that may be required when utilizing active devices to provide vacuum pressure to return the room air samples. As disclosed, the sample return unit 114 may utilize the existing fluid pressure from the HVAC system 116 to power the sample return unit 114 via the Venturi effect to improve efficiency and limit maintenance.

Though the sample return unit 114 is described in the exemplary embodiment as a Venturi vacuum device, in some embodiments, the sample return unit 114 may recover the room air samples from the sensor pack outlet 80 and the multiplexor purge outlet 58 utilizing conventional vacuum pumps or fans. For example, in some embodiments, the sample return unit 114 may utilize suction created via a reciprocating pump, screw or turbine compressor, or various devices configured to displace air in response to receiving electrical current. Such devices may be utilized to provide vacuum pressure to return the room air samples. However, these devices may be noisy, inefficient, and require more maintenance than the Venturi vacuum device disclosed herein.

By continuously priming the subsequent channel of the room to be measured next, i.e., the primed second or primed channel room 109 (e.g. room 12), by purging the sample from the same via the purge airflow line 123, the respective supply line 120 is clear of old or outdated air samples for the subject room within the supply line 120 when the second or primed channel room 109 (e.g., room 14) having its inlet 106b aligned with the second inlet core 64 and the purge airflow line 123 is transitioned to the measured volumetric region 108 having its inlet 106b aligned with the first core inlet 62 and the sensor airflow line 121. Said another way, once the channel is primed, via the purge airflow line 123, the respective supply line 120 contains an air sample that is representative of a current air quality in the respective room 102.

One challenge in implementing air quality or particulate sensors may relate to variations in system operation, which may limit accuracy. To address such potential variations, particularly in flow rate or fluid delivery from remotely located rooms or regions and maintain the measurement or detection accuracy of the system, the disclosure provides for a monitoring routine with a dynamic dwell time.

In particular, the calculation of the dynamic dwell time, or cycle time T_{c} that the rotatable core spends in any one position, i.e., evaluating a respective measured volumetric region 108 may include taking a sum of a measurement time Tₘ (the time needed for a respective sensor 140 to take a reading on the air sample 132) and a calculated purge time Tₚ that may vary proportionally to the flow rate of each air sample 132 delivered to the sensor pack inlet 78 as measured by an operational sensor 150 that is an airflow sensor disposed at the sensor pack inlet 78.

The monitoring routine 300 demonstrated in FIG. 8 provides for an exemplary procedure for adjusting the cycle time T_{c} among the rooms 102 or regions, based on the sensed air flow rate of the respective air sample 132 as detected at the sensor pack inlet 78 to improve the measurement accuracy of the system 100.

As demonstrated throughout FIGS., an air sample from the selected room or measured volumetric region 108 may be delivered to the remote test environment 122 via the sensor airflow line 121. By way of example, the controller 118 may output a control signal to the power unit 76 to rotate the rotatable core 60 to a first position, such that the first core inlet 62 is aligned with the first multiplexor inlet 106a, that is associated with the room for sensing or measurement or the measured volumetric region 108 (e.g., room 12), such that the air sample corresponding to the measured volumetric region 108 (e.g., room 12), is conveyed to the sensor pack inlet 78 via the sensor airflow line 121.

The air flow rate of the respective air sample 132 corresponding to the measured volumetric region 108 (e.g., room 12), at the sensor pack inlet 78 may be sensed by an operation sensor 150 defined as an airflow sensor, such that the operation sensor 150 generates an airflow rate reading. The operation sensor 150 then transmits the respective airflow rate reading to the controller 118.

In the example shown, after receiving the respective air flow rate reading from the operation sensor 150, the controller 118 may calculate a cycle time T_{c} based on the sensed air flow rate for the selected region 108. In one example, the controller 118 may concurrently calculate the purge time Tₚ, measurement time Tₘ, and cycle time T_{c} based on the sensed airflow rate. Alternatively, it shall be understood that, the controller 118 may calculate the purge time Tₚ, measurement time Tₘ, and cycle time T_{c} for the selected region 108 or room sequentially, wherein the controller 118 first calculates the purge time Tₚ and measurement time Tₘ for the selected region 108 or room, and then calculates the cycle time T_{c} for the selected region 108 or room by combining or taking a sum of the calculated purge time Tₚ and measurement time Tₘ.

More particularly, the measurement time Tₘ may be determined based on the time required to detect air quality characteristics or particle content based for each of the sensors 140 at the detected flow rate as previously discussed. The purge time Tₚ may be calculated as the time required to displace a known volume of the air channel 126 at the detected flow rate after the measurement time Tₘ is complete.

The cycle time T_{c} is likely to vary amongst rooms or regions. For example, the duration required to purge air from the sensor airflow line 121 (purge time Tₚ) may vary widely depending on the location of the selected room or region and the resultant flow rate at which the air sample 132 from the selected room or region is delivered to the sensor pack inlet 78, due to variations in the tubing of the supply lines 120, including the length, style, materials, bends, routing, wear, and/or clogging from debris, and other frictional losses.

As such, when the controller 118 outputs a control signal to the power unit 76 to rotate the rotatable core 60 from a first position to a second position, such that the first core inlet 62 is aligned with the second multiplexor inlet 106b, that is associated with the room for sensing or measurement or the measured volumetric region 109 (e.g., room 14), such that the air sample corresponding to the measured volumetric region 109 (e.g., room 14), is conveyed to the sensor pack inlet 78 via the sensor airflow line 121. The air flow rate of the respective air sample 132 corresponding to the measured volumetric region 109 (e.g., room 14), will be different than that of volumetric region 109 (e.g., room 12).

Said another way, the duration required to purge air from the sensor airflow line 121 or the purge airflow line 123 that has been delivered from room 14 (FIG. 1) will be longer than the duration required to purge air from the sensor airflow line 121 and the purge airflow line 123 that has been delivered from room 12 (FIG. 1) due to increased frictional and other losses to the air flow rate that will be incurred by the sample 132 from the more distant Room 14, thereby resulting in a decreased delivery air flow rate of the air sample 132, from Room 14 as compared to Room 12, to the sensor pack inlet 78.

To account for these variations in operation, the controller 118 may monitor the flow rate among the air samples delivered from the rooms or regions 102 and adjust the cycle time T_{c} (time the rotatable core 60 spending in any one position, e.g., the first position, the second position, etc.) to compensate for the variations in air flow rate. In this way, the system 100 may dynamically adjust the time required to detect conditions representative of the selected room, i.e., the measurement time T m, during which the air sample 132 is contained in the remote test environment 122 and dynamically adjust the time required to purge the respective sample from the sensor airflow line 121, i.e., the purge time Tₚ.

Once calculated, the purge time Tₚ, measurement time Tₘ, cycle time T_{c} and/or the flow rate for the selected room or region, may be stored in a memory or database. As the system 100 cycles through each of the rooms or regions 102, the purge time Tₚ, measurement time Tₘ, and/or flow rate data may be stored and updated to a memory according to a predetermined schedule or update frequency (e.g., every cycle, daily, weekly, monthly, etc.). In this way, the controller 118 may detect the flow rate for each of the selected rooms or regions 108 throughout the operation of the system 100 and adjust the cycle time T_{c} for each of the corresponding sample periods to maintain accurate measurements despite variations in the flow rate. Additionally, or alternatively, the purge time Tₚ and/or flow rate of each of the air samples delivered from the sample rooms or regions 102 may be stored to the memory through a periodic testing or calibration procedure that may be completed at scheduled intervals or during the setup of the system 100.

As such in operation, upon delivery of the room air sample from the selected room or region 108 (e.g., room 12), the controller 118 may to purge the air from the air channel 126 along the sensor airflow line 121 over the purge time Tₚ as the rotatable core 60 is moved from the first position to the second position. Concurrently, the operation sensor 150 may detect a flow rate of the air sample 132 from the selected room or region 109 (e.g., room 14). Though demonstrated as detecting the updated flow rate while concurrently purging the air from remote testing environment 122, the purge time Tₚ may be initiated sequentially following the detection of the flow rate.

Once the purge time Tₚ has elapsed for the air sample 132 from the selected room or region 108 (e.g., room 12), the controller 118 may facilitate the movement of the air sample 132 from the selected room or region 109 (e.g., room 14) through the sensor airflow line 121 and into the air channel 126. Once the air sample 132 from the selected room or region 109 (e.g., room 14) is present in the remote testing environment 122, the controller 118 may initiate the detection routine during the measurement time Tₘ for the one or more sensors 140 implemented in remote testing environment 122 to evaluate the respective air quality metric.

Following the measurement time Tₘ required for the one or more sensors 140 to evaluate the respective air quality metric, the controller 118 may read out or receive sensor data from each of the sensors 140. Following the successful determination of the sensor data from each of the sensors 140, the controller 118 may continue the monitoring routine by assigning the next room or region 102 as the selected room or region to continue to sequentially monitor the air quality or air characteristics of each of the regions or rooms 102 in fluid communication with the remote testing environment 122.

With the cycle time T_{c}, including the dynamic purge time Tₚ, being calculated as a function of the air flow rate sensed by the operation sensor 150 at the sensor pack inlet 78, the system 100 may continue the monitoring routine as described throughout the disclosure and reduce overall system cycle time, i.e., the time to cycle through measurement of all rooms or regions 102.

It will be appreciated that embodiments of the disclosure described herein may be comprised of one or more conventional processors or controllers with stored program instructions that control one or more processors to implement, in conjunction with certain non-processor circuits, some, most, or all of the functions of an image sensor system and method thereof, as described herein. The non-processor circuits may include, but are not limited to, signal drivers, clock circuits, power source circuits, and/or user input devices. Alternatively, some or all functions could be implemented by a state machine that has no stored program instructions, or in one or more application specific integrated circuits (ASICs), in which each function or some combinations of the functions are implemented as custom logic. Of course, a combination of the two approaches could be used. Thus, the methods and means for these functions have been described herein. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and integrated circuits with minimal experimentation.

The detailed description and the drawings or figures are supportive and descriptive of the present teachings, but the scope of the present teachings is defined solely by the claims. While some of the best modes and other embodiments for carrying out the present teachings have been described in detail, various alternative designs and embodiments exist for practicing the present teachings defined in the appended claims.

While various embodiments have been described, the description is intended to be exemplary, rather than limiting and it will be apparent to those of ordinary skill in the art that many more embodiments and implementations are possible that are within the scope of the embodiments. Any feature of any embodiment may be used in combination with or substituted for any other feature or element in any other embodiment unless specifically restricted. Accordingly, the embodiments are not to be restricted except in light of the attached claims and their equivalents. Also, various modifications and changes may be made within the scope of the attached claims.

Benefits, other advantages, and solutions to problems, and any element or elements that may cause any benefit, advantage, or solution to occur or become more pronounced, however, are not to be construed as critical, required, or essential features or elements of any or all of the claims, unless such benefits, advantages, solutions, or elements are expressly stated in such claims.

What is claimed is:

1. An air quality monitoring system configured to detect an air quality metric for a plurality of volumetric regions, the system comprising:
a multiplexor configured to receive a plurality of air samples from each of the plurality of volumetric regions, the multiplexor comprising:
a multiplexor housing having a base that defines a multiplexor sensor outlet and a cover that defines a plurality of multiplexor inlets in fluid communication with the plurality of volumetric regions, wherein the plurality of multiplexor inlets comprises a first multiplexor inlet that corresponds a measured volumetric region;
a rotatable core defining a first core inlet;
a power unit in electrical communication with the rotatable core and configured to rotate the rotatable core, such that the first core inlet is aligned with the inlet associated with the measured volumetric region;
a sensor pack comprising:
a sensor pack housing defining an interior cavity and a sensor pack inlet in fluid communication with the multiplexor sensor outlet and a sensor pack outlet; and
at least one sensor disposed within the interior cavity, the at least one sensor configured to measure an air quality metric of the selected air sample;
a sensor airflow line collectively defined by the multiplexor and the sensor pack, the sensor airflow line configured to fluidly couple one of the volumetric region inlets, the first core inlet, the multiplexor sensor outlet, the sensor pack inlet, the interior cavity, and the sensor pack outlet; and
a controller in electrical communication with each of the power unit and the sensor pack, the controller configured to direct the power unit to actuate the multiplexor rotatable core between a plurality of positions to thereby align the respective multiplexor inlet with the sensor airflow line.

2. The air quality monitoring system of claim 1 wherein:
the plurality of multiplexor inlets further comprises a second multiplexor inlet associated with a primed volumetric region;
the base further defines a purge outlet; and
the rotatable core further defines a second core inlet.

3. The air quality monitoring system of claim 2, the multiplexor further comprising:
a bypass structure having a bypass inlet and a bypass outlet; and
a purge airflow line configured to fluidly couple the second multiplexor inlet, the second core inlet, the bypass inlet, the bypass outlet, and the purge outlet.

4. The air quality monitoring system of claim 3 wherein when the controller directs the power unit to actuate the rotatable core to the first position, the second multiplexor inlet is aligned with the second core inlet, such that the air sample corresponding to the primed volumetric region is discarded via the purge airflow line.

5. The air quality monitoring system of claim 1 wherein the controller is further configured to:
direct the power unit to actuate the multiplexor rotatable core to a first position that aligns the first multiplexor inlet with the sensor airflow line, such that the air sample for the measured volumetric region is directed to the sensor pack for evaluation;
receive a signal from the at least one sensor indicating the measured air quality metric of the air sample corresponding to the measured volumetric region; and
signal the power unit to rotate the multiplexor core to a second position to change the volumetric region with which the first core inlet is aligned, such that the air quality metric is identified for each of the plurality of regions.

6. The air quality monitoring system of claim 5, wherein when the controller directs the power unit to actuate the rotatable core to the second position wherein the second multiplexor inlet is aligned with sensor airflow line, such that the air sample for the primed volumetric region is directed to the sensor pack for evaluation.

7. The air quality monitoring system of claim 6 wherein the plurality of multiplexor inlets further comprises a third multiplexor inlet associated with a third volumetric region, and wherein when the controller directs the power unit to actuate the rotatable core to the second position, the third multiplexor inlet is aligned with the second core inlet, such that the air sample corresponding to the third volumetric region is discarded via the purge airflow line.

8. The air quality monitoring system of claim 5 wherein the multiplexor further comprises a magnetic encoder configured to evaluate the relative position of the rotatable core and transmit a core position signal to the controller.

9. The air quality monitoring system of claim 8 wherein each of the multiplexor housing and rotatable core are comprised of a chemically inert material.

10. The air quality monitoring system of claim 9 wherein the cover comprises a Polytetrafluoroethylene (PTFE) material.

11. The air quality monitoring system of claim 9 wherein the base comprises an acrylonitrile butadiene styrene (ABS) material.

12. The air quality monitoring system of claim 9 wherein the rotatable core comprises a Polytetrafluoroethylene (PTFE) material.

13. The air quality monitoring system of 9 wherein the sensor pack housing comprises a housing base and a housing cover and wherein the at least one sensor comprises a first plurality of sensors and a second plurality of sensors.

14. The air quality monitoring system of claim 13 wherein:
the first plurality of sensors comprises at least one of at least one of a volatile organic compound (VOC) sensor, a particulate matter sensor, a relative humidity sensor, a carbon dioxide sensor, a carbon monoxide sensor, an ozone sensor, an ammonia sensor, a formaldehyde sensor, a temperature sensor, a pressure sensor, and a velocity sensor; and
the second plurality of sensors comprises at least one of at least one of a volatile organic compound (VOC) sensor, a particulate matter sensor, a relative humidity sensor, a carbon dioxide sensor, a carbon monoxide sensor, an ozone sensor, an ammonia sensor, a formaldehyde sensor, a temperature sensor, a pressure sensor, and a velocity sensor.

15. The air quality monitoring system of 14 wherein the sensor pack further comprises a substrate disposed between the first plurality of sensors and the second plurality of sensors, wherein the substrate defines an air channel therein that extends from the sensor pack inlet to the sensor pack outlet.

16. The air quality monitoring system of 15 wherein the first plurality of sensors is disposed on a first sensor board having a first board sensing surface is in fluid communication with the air channel and a first board auxiliary surface disposed in contact with the housing base.

17. The air quality monitoring system of claim 16 wherein the second plurality of sensors is disposed on a second sensor board having a sensing surface in fluid communication with the air channel and a second board auxiliary surface disposed in contact with the housing cover.

18. The air quality monitoring system of claim 17 wherein:
the first sensor board comprises a plurality of modular board segments; and
the second sensor board comprises a plurality of modular board segments.

19. The air quality monitoring system of claim 18 wherein:
each modular board segment comprises of the first sensor board comprises one of the sensors of the first plurality of sensors; and
each modular board segment the second sensor board comprises one of the sensors of the second plurality of sensors.

20. The air quality monitoring system of claim 19 further comprising a plurality of air sample return units, and wherein:
the plurality of air sample return units comprises at least a first air sample return unit disposed between the sensor pack outlet and a building ventilation system and a second air sample return unit disposed between the purge outlet and the building ventilation system; and
each of the air sample return units are Venturi vacuum devices.
